(19) United States
(12) Patent Application Publication
(10) Pub. No.: US 2025/0327730 A1
(43) Pub. Date: Oct. 23, 2025

(54) METHOD FOR INVERTING TRUE MASS CONTENT OF WATER ICE IN LUNAR SOIL USING LUNAR SOIL WATER MOLECULE ANALYZER

(71) Applicant: Hefei Institutes of Physical Science, CAS, Hefei (CN)

(72) Inventors: Ruifeng KAN, Hefei (CN); Nailiang CAO, Hefei (CN); Xiang LI, Hefei (CN); Zhenyu XU, Hefei (CN); Xingping WANG, Hefei (CN); Wenzhen LU, Hefei (CN); Chi ZHANG, Hefei (CN)

(73) Assignee: Hefei Institutes of Physical Science, CAS, Hefei (CN)

(21) Appl. No.: 19/186,679

(22) Filed: Apr. 23, 2025

(30) Foreign Application Priority Data
Apr. 23, 2024 (CN) 202410492770.6

(51) Int. Cl. G01N 7/16; G01N 33/24

(52) U.S. Cl. CPC G01N 7/16; G01N 33/246

(57) ABSTRACT

A method for inverting true mass content of water ice in lunar soil using a lunar soil water molecule analyzer (LSWMA) includes: obtaining weight data of a lunar soil sample, temperature data of a sample receiving container that receives the lunar soil sample, and duration of a transfer process of the lunar soil sample; obtaining measurement data of total pressure of the water vapor; calculating a sublimation loss based on a sublimation rate and the duration of the transfer process, and estimating a relative sublimation loss rate; correcting the measurement data of the total pressure of the water and obtaining true pressure data of the water vapor; and calculating, based on the true pressure data of the water vapor, mass of the water vapor, and further calculating mass content of water ice in lunar soil.

### CROSS REFERENCE TO THE RELATED APPLICATIONS

This application is based upon and claims priority to Chinese Patent Application No. 202410492770.6, filed on Apr. 23, 2024, the entire contents of which are incorporated herein by reference.

### TECHNICAL FIELD

The present disclosure relates to the field of inversion measurement of water content in lunar soil, and specifically, to a method for inverting true mass content of water ice in lunar soil using a lunar soil water molecule analyzer (LSWMA).

### BACKGROUND

The occurrence form and content of water ice in a permanently shadowed region (PSR) of a lunar polar region have always been challenging issues for water ice exploration in the lunar polar region, and will also be one of key scientific goals of China's Chang'e-7 lunar exploration program. So far, main in-situ lunar water ice exploration missions planned or conducted at abroad are NASA's Volatiles Investigating Polar Exploration Rover (VIPER) and Russia's Luna 25/27. The VIPER will carry a Near-Infrared Volatile Spectrometer System (NIRVSS) to perform inversion measurement on water content in lunar soil on a lunar surface and profile through water ice reflection spectra. However, measurement accuracy of water mass content is not high due to influence of multiple variables such as a particle size of the lunar soil and a temperature of the lunar surface.

The European Space Agency (ESA) plans to deploy a ProSPA volatile analyzer on the Luna 27, which employs a method of drilling, heating, and extracting the lunar soil. In this method, drilled lunar soil is transferred to a heating device for heating, such that water ice in the lunar soil is converted into water vapor, and then the inversion measurement is performed on the water vapor via a magnetic mass spectrometry device in the ProSPA volatile analyzer. This method requires a high heating temperature and achieves nearly 100% water ice extraction efficiency, but fails to quantify a sublimation loss during lunar soil sample collection and transfer, causing a large error in calculating the in situ mass content of water ice in original lunar soil.

China's “Chang'e-7” LSWMA also uses the method of drilling, heating, and extracting the lunar soil to perform the inversion measurement on content of the water ice in the lunar soil. Specifically, the drilled lunar soil is placed in a constant-volume sample receiving cup of the LSWMA, and then transferred from the sample receiving cup to a vacuum furnace chamber of the LSWMA. A heating capability of a Metal Ceramic Heater (MCH) of the sample receiving cup is used to heat the lunar soil to convert the water ice in the lunar soil into the water vapor. All the water vapor enters a spectral unit of the LSWMA through a pipeline, and water molecule molar concentration is measured by the spectral unit. Total pressure of the gas in the spectral unit is measured by a vacuum gauge. The water ice content in lunar regolith is determined by reconstructing the measured water vapor pressure to its original total pressure prior to sublimation, adsorption, and other loss processes, calculating the initial water vapor mass from this reconstructed pressure. Then the content of the water ice in the lunar soil is calculated based on the weight of the water ice and a weight of the lunar soil. Sublimation during regolith sample collection and transfer induces water vapor loss, leading to significant errors in the water ice content inversion by the LSWMA. Furthermore, water vapor adsorption in pipelines and gas leakage from the interconnected pipeline-spectral unit system compound vapor loss, exacerbating inversion error in the content of the water ice by the LSWMA.

### SUMMARY

The present disclosure provides a method for inverting true mass content of water ice in lunar soil using a lunar soil water molecule analyzer (LSWMA), in order to avoid a large error in an inversion result when inversion measurement is performed on content of water ice in lunar soil by means of drilling, heating, and extracting the lunar soil in the prior art.

To achieve the above objective, the present disclosure adopts following technical solutions:
A method for inverting true mass content of water ice in lunar soil using a LSWMA is provided, including: obtaining weight data of a lunar soil sample, and when a sample receiving container of a LSWMA receives the lunar soil sample, obtaining temperature data of the sample receiving container that receives only the lunar soil sample; in a process of transferring the lunar soil sample from the sample receiving container to a vacuum container by the LSWMA, obtaining duration of the entire transfer process; then, performing vacuum heating on the lunar soil sample in the sample receiving container by using the vacuum container, such that water ice in the lunar soil sample is all converted into water vapor, and when the water vapor enters a spectral unit through a pipeline, obtaining measurement data of total pressure of the water vapor in the spectral unit;
determining a sublimation rate of water ice in the lunar soil sample based on the temperature data of the sample receiving container, calculating a sublimation loss of the water ice in the transfer process based on the duration of the transfer process, and then estimating a relative sublimation loss rate of the water ice in the lunar soil sample in the transfer process based on the sublimation loss;
correcting the measurement data of the total pressure of the water vapor in the spectral unit based on the relative sublimation loss rate, pre-measured water vapor extraction efficiency during the vacuum heating performed by the vacuum container on the lunar soil sample, a relationship between a relative adsorption rate of the pipeline for the water vapor and pressure measurement data of the water vapor, and a total system leakage of the LSWMA, and obtaining true pressure data of the water vapor; and
calculating, based on the true pressure data of the water vapor, mass of the water vapor converted from the water ice in the lunar soil sample, namely mass of the water ice in the lunar soil sample, and calculating mass content of the water ice in the lunar soil sample based on the mass of the water ice and the weight data of the lunar soil sample.

Further, based on the temperature data of the sample receiving container, the sublimation rate S of the water ice is calculated using a calculation formula for a sublimation rate of pure water ice, as shown in a formula (1):

$$S = {e}_{s a t , i} ( T ) \times \sqrt{\frac{{M}_{w}}{2 π R T}} \times exp ( \frac{2 {M}_{w} {σ}_{i}}{{ρ}_{i} R T} ) \qquad ( 1 )$$

where in the formula (1), e_{sat,i}(T) represents saturated vapor pressure of water, and is obtained by calculating a temperature of the lunar soil sample based on the temperature data of the sample receiving container and then substituting the temperature of the lunar soil sample into an empirical formula shown in a formula (2):

$${e}_{s a t , i} = exp ( 9.550426 - \frac{5 723.265}{T} + 3.53068 ln T - 0.00728332 T ) ; \qquad ( 2 )$$

M_{W} represents molar mass of a water molecule, and is 18.015×10⁻³ kgmol⁻¹;
R represents a universal gas constant, and is 8.31447 Jmol⁻¹K⁻¹;

$$exp ( \frac{2 {M}_{w} {σ}_{i}}{{ρ}_{i} R T} )$$

represents a correction term for saturated vapor pressure generated by the water ice due to surface tension under a true condition, where σᵢ represents surface tension on a pure ice/vapor interface, with a value of 0.109 Jm⁻²; and ρᵢ represents a density of the water ice at a sublimation temperature, and a calculation formula for the ρᵢ is shown in a formula (3):

$${ρ}_{i} = 916.7 - 0.175 ( T - 273.15 ) - 5.0 \times 1 {0}^{- 4} ( T - 273.15 ) ; \qquad ( 3 )$$

in the above formulas (1), (2), and (3), T is taken as the temperature that is of the lunar soil sample and calculated based on the temperature data of the sample receiving container;
then, a theoretical sublimation loss of the water ice in the transfer process is calculated according to a following formula: A=S×t₁, where t₁ represents the duration of the transfer process;
next, the theoretical sublimation loss is corrected based on a correction coefficient k that is related to a particle size and a pore of the lunar soil sample and obtained from a pre-experiment, and an actual sublimation loss is obtained according to a following formula: A₁=A×k; and
finally, based on the actual sublimation loss A₁ and an original water amount m1 corresponding to the correction coefficient k during the pre-experiment, the relative sublimation loss rate is calculated according to a following formula:

$$a = \frac{A 1}{m 1} .$$

Further, a process for determining the correction coefficient k based on an experiment is as follows: during the experiment, taking a plurality of lunar soil simulants that have different porosity and particle sizes and have a same water content of mass m1, placing the lunar soil simulants in the sample receiving container, and obtaining sublimation losses of each of the lunar soil simulants within a plurality of different temperature ranges in the sample receiving container; and obtaining sublimation losses of pure water ice with mass of m1 within a plurality of corresponding temperature ranges in the sample receiving container, and determining the correction coefficient k based on the sublimation losses of the lunar soil simulants and the sublimation losses of the pure water ice.

Further, when the water vapor extraction efficiency during the vacuum heating performed by the vacuum container on the lunar soil sample in the sample receiving sample is measured, a plurality of lunar soil simulants that have a same original weight but different moisture contents are taken and separately placed in a sample receiving container of the vacuum container for heating, weight data of each of the lunar soil simulants during the heating is recorded, and then the recorded weight data of each of the lunar soil simulants is nonlinearly fitted to obtain the water vapor extraction efficiency during the vacuum heating performed by the vacuum container on the lunar soil sample.

Further, when the water vapor extraction efficiency is measured, weight data of the sample receiving container in empty state during the heating is also recorded, and after a weight change of the sample receiving container in empty state due to the heating is deducted, nonlinear fitting is performed to obtain the water vapor extraction efficiency when the vacuum container performs the vacuum heating on the lunar soil sample.

Further, when the relationship between the relative adsorption rate of the pipeline for the water vapor and the pressure measurement data of the water vapor is determined, a given amount of water is taken and converted into constant-pressure water vapor of a plurality of gradients, and constant-pressure water vapor of each of the gradients is separately injected into the spectral unit through the pipeline; a pressure change of the constant-pressure water vapor of each of the gradients in the spectral unit due to pipeline adsorption, as well as pressure that is of the water vapor in the spectral unit and no longer decreases after the pipeline adsorption is saturated, namely equilibrium pressure, are recorded, and a relative adsorption rate of the constant-pressure water vapor of each of the gradients is obtained based on the pressure change and the equilibrium pressure; and then, based on fitted pressure of the constant-pressure water vapor of the gradients, a corresponding relationship between the relative adsorption rate and pressure data that is of the water vapor and measured by a vacuum gauge for the spectral unit is obtained, that is, the relationship between the relative adsorption rate of the pipeline for the water vapor and the pressure measurement data of the water vapor is obtained.

Further, when the total system leakage of the LSWMA is measured, a constant-pressure gas is injected into a connected system constituted by the vacuum container, the pipeline, and the spectral unit in the LSWMA, a curve reflecting that pressure in the spectral unit changes over time is recorded, then a system leakage rate is obtained by using a static pressure rise method based on the curve, and finally, the total system leakage is calculated based on the system leakage rate and total time corresponding to the curve.

Further, the measurement data of the total pressure of the water vapor in the spectral unit is corrected according to a following formula (6):

$$P c = X + Pm / ( ( 1 - a ) * η * ( 1 - b ) ) \qquad ( 6 )$$

where in the formula (6), Pc represents the true pressure data of the water vapor; Pm represents the measurement data of the total pressure of the water vapor in the spectral unit; X represents the total system leakage of the LSWMA; a represents the relative sublimation loss rate; η represents the water vapor extraction efficiency during the vacuum heating performed by the vacuum container on the lunar soil sample; b represents the relative adsorption rate of the pipeline for the water vapor, where the relative adsorption rate b of the pipeline for the water vapor is calculated by substituting the measurement data of the total pressure of the water vapor in the spectral unit into the relationship between the relative adsorption rate of the pipeline for the water vapor and the pressure measurement data of the water vapor.

Further, based on the true pressure data of the water vapor, a volume of the spectral unit, and a measured temperature of the spectral unit, the mass of the water vapor converted from the water ice in the lunar soil sample is calculated using an ideal gas law.

The present disclosure fully considers a water vapor loss when a LSWMA performs inversion measurement on content of water ice by means of drilling, heating, and extracting lunar soil, including a sublimation loss of transferring the lunar soil from a sample receiving container to a vacuum tank, an adsorption loss caused by absorption of a pipeline for water vapor when the water vapor passes through the pipeline, and a leakage loss of a connected system constituted by the vacuum container, the pipeline, and a spectral unit in the LSWMA. Water vapor extraction efficiency during vacuum heating performed by the vacuum container on a lunar soil sample is also fully considered. Therefore, a compensation term is constructed to compensate for measurement data of total pressure of the water vapor in the spectral unit to obtain true pressure data of the water vapor. The true pressure data of the water vapor can more truly reflect pressure of water vapor that has not been sublimated, adsorbed, or leaked in the spectral unit. Therefore, mass of the water vapor, which is calculated based on the true pressure data of the water vapor, is closer to a true weight of the water ice in the lunar soil, which can reduce an error of finally calculated content of the water ice in the lunar soil.

According to the method in the present disclosure, sensitivity of the LSWMA for detecting the content of the water ice in the lunar soil is better than 0.1 wt %, providing reliable data support for evaluation of a total amount of water ice resources in lunar poles by Chang'e-7.

### BRIEF DESCRIPTION OF THE DRAWINGS

FIG. 1 is a schematic diagram of a method according to an embodiment of the present disclosure; and

FIG. 2 shows a fitted curve of a relationship between a relative adsorption rate of a pipeline for water vapor and pressure measurement data of the water vapor according to an embodiment of the present disclosure.

### DETAILED DESCRIPTION OF THE EMBODIMENTS

To enable those skilled in the art to better understand the solutions in the present disclosure, the following describes implementations of the present disclosure in detail with reference to the accompanying drawings and embodiments in the present disclosure, to sufficiently understand and execute a realization process of solving technical problems through technical means and achieving corresponding technical effects. The embodiments of the present disclosure and all features in the embodiments can be mutually combined under the premise of no existence of conflict, and a formed technical solution falls within the protection scope of the present disclosure.

Apparently, the described embodiments are only a part of, not all of, the embodiments of the present disclosure. All other embodiments obtained by a person of ordinary skill in the art based on the embodiments of the present disclosure without creative efforts shall fall within the protection scope of the present disclosure.

It should be noted that the terms “comprising/including”, “having”, and any variations thereof in the specification and claims of the present disclosure are intended to cover non-exclusive inclusion.

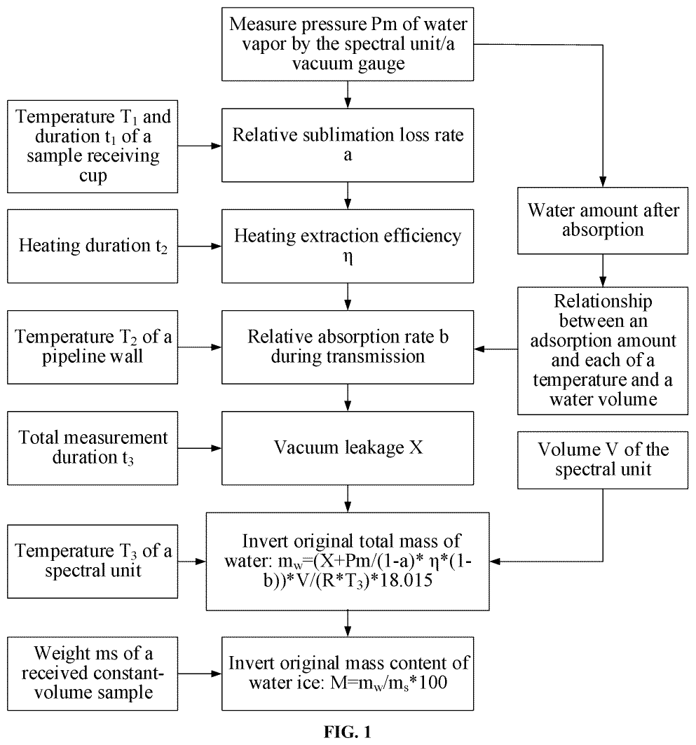

As shown in FIG. 1, an embodiment provides a method for inverting true mass content of water ice in lunar soil using a LSWMA. A process of the method in this embodiment is further described below in conjunction with a working process of the LSWMA.

In this embodiment, when a drilled lunar soil sample is completely sent to a sample receiving container (namely a constant-volume sample receiving cup) of the LSWMA, it is difficult to weigh the lunar soil sample under ⅙ gravity of the moon. Therefore, the constant-volume sample receiving cup is used in this embodiment, and weight data ms of the lunar soil sample is determined based on a density and a volume of naturally accumulated lunar soil samples in the constant-volume sample receiving cup. A density of lunar soil can be obtained through a simulation experiment in advance based on funnel sample collection conditions of a gas extraction unit of the LSWMA.

In this embodiment, after the constant-volume sample receiving cup receives the complete lunar soil sample, temperature data T₁ of the constant-volume sample receiving cup that receives the complete lunar soil sample is measured. Specifically, the constant-volume sample receiving cup is an MCH, which uses alumina ceramic and a built-in electric heating wire. The temperature data T₁ is obtained by applying a direct current to the heating wire of the constant-volume sample receiving cup and measuring a resistance value returned by the alumina ceramic.

In this embodiment, when the LSWMA transfers the lunar soil sample in the constant-volume sample receiving cup to a constant-volume sample receiving cup in a vacuum container (namely a vacuum tank), duration t₁ of the entire transfer process is recorded.

Since a water molecule in the lunar soil sample at this time exists in a form of water ice, a sublimation rate S of the water ice is calculated using a calculation formula for a sublimation rate of pure water ice in this embodiment. The calculation formula for the sublimation rate of the pure water ice is shown in a formula (1):

$$S = {e}_{s a t , i} ( T ) \times \sqrt{\frac{{M}_{w}}{2 π R T}} \times exp ( \frac{2 {M}_{w} {σ}_{i}}{{ρ}_{i} R T} ) \qquad ( 1 )$$

In the formula (1):
e_{sat,i}(T) represents saturated vapor pressure of water at a sublimation temperature parameter T, in units of Pa, where within a range of 110 K to 273 K, the e_{sat,i}(T) is calculated according to an empirical formula, and the empirical formula is shown in a formula (2):

$${e}_{s a t , i} = exp ( 9.550426 - \frac{5 723.265}{T} + 3.53068 ln T - 0.00728332 T ) ; \qquad ( 2 )$$

M_{W} represents molar mass of the water molecule, and is 18.015×10⁻³ kg·mol⁻¹;
R represents a universal gas constant, and is 8.31447 Jmol⁻¹K⁻¹; and

$$exp ( \frac{2 {M}_{w} {σ}_{i}}{{ρ}_{i} R T} )$$

represents a correction term for saturated vapor pressure generated by the water ice at the sublimation temperature parameter T due to surface tension under a true condition, where σᵢ represents surface tension on a pure ice/vapor interface, with a value of 0.109 Jm⁻²; ρᵢ represents a density of the water ice at the sublimation temperature parameter T, in units of Kgm⁻³; and a calculation formula for the ρᵢ is shown in a formula (3):

$${ρ}_{i} = 916.7 - 0.1 \; 75 ( T - 273.15 ) - 5.0 \times 1 {0}^{- 4} ( T - 273.15 ) . \qquad ( 3 )$$

In specific calculation, due to low thermal conductivity of the lunar soil, a temperature of the constant-volume sample receiving cup does not reflect a true temperature of the lunar soil sample. Therefore, it is necessary to calculate the temperature Tn of the lunar soil sample based on the temperature data T₁ of the constant-volume sample receiving cup and the thermal conductivity of the lunar soil. A model proposed by KSchreiner et al. (2016) for calculating thermal characteristics of a 350 K or more than 350 K silicate mineral in the lunar soil (Schreiner, Samuel S., et al. “Thermal property models for lunar regolith.” Advances in space research 57.5 (2016): 1209-1222) is introduced during the calculation. COMSOL is used to model heat transfer inside the constant-volume sample receiving cup, and a temperature value is obtained through solving.

After the temperature Tn of the lunar soil is obtained, the temperature Tn of the lunar soil is substituted into the formula (3) to obtain the ρᵢ, and then a correction term at the temperature Tn of the lunar soil can be obtained by substituting the obtained ρᵢ into the

$$exp ( \frac{2 {M}_{w} {σ}_{i}}{{ρ}_{i} R T} )$$

and setting the T to be equal to the temperature Tn of the lunar soil. In addition, saturated vapor pressure at the temperature Tn of the lunar soil can be obtained by substituting the temperature Tn of the lunar soil into the formula (2).

Finally, the temperature Tn of the lunar soil is substituted into the sublimation temperature parameter T in the formula (1), and the calculated correction term and saturated vapor pressure at the temperature Tn of the lunar soil are separately substituted into the formula (1) to calculate the sublimation rate S of the water ice in the lunar soil sample received by the constant-volume sample receiving cup. The sublimation rate S is multiplied by the duration t₁ of the transfer process to obtain a theoretical sublimation loss A of the water ice in the transfer process, namely A=S×t₁.

Considering that a porous structure of real lunar soil affects the sublimation rate of the water ice, this embodiment introduces a correction coefficient k related to a particle size and a pore of the lunar soil to correct the calculated theoretical sublimation loss A. The correction coefficient k is obtained from a pre-experiment. Specifically, during the experiment, a plurality of lunar soil simulants that have different porosity and particle sizes and have a same water content of mass m1 are taken and placed in the constant-volume sample receiving cup, and sublimation losses of each lunar soil simulant within a plurality of different temperature ranges (T11-T12, T12-T13, T13-T14, . . . ) in the constant-volume sample receiving cup are obtained. In addition, pure water ice with mass of m1 is taken and placed in the constant-volume sample receiving cup, and sublimation losses of the pure water ice within a plurality of corresponding temperature ranges (T11-T12, T12-T13, T13-T14, . . . ) in the sample receiving container are obtained. The correction coefficient k is determined based on the sublimation losses of the lunar soil simulants and the sublimation losses of the pure water ice.

In this embodiment, the theoretical sublimation loss A is obtained. Then an actual sublimation loss A₁ is calculated based on the theoretical sublimation loss A and the correction coefficient k obtained from the pre-experiment, namely A₁=A×k. Then, a relative sublimation loss rate a is calculated based on the actual sublimation loss A₁ and an original water amount corresponding to the correction coefficient k, namely the m1. A calculation formula for the relative sublimation loss rate a is as follows:

$$a = \frac{A 1}{m 1} .$$

In this embodiment, when the LSWMA performs vacuum heating on the lunar soil sample in the constant-volume sample receiving cup through the vacuum container to convert the water ice in the lunar soil sample to water vapor, and the water vapor enters a spectral unit through a pipeline, measurement data Pm of total pressure of the water vapor in the spectral unit is inverted by the spectral unit or obtained by using a configured vacuum gauge.

The measurement data Pₘ of the total pressure of the water vapor in the spectral unit is corrected based on the relative sublimation loss rate a, pre-measured water vapor extraction efficiency η during the vacuum heating performed by the vacuum container on the lunar soil sample, a relationship between a relative adsorption rate b of the pipeline for the water vapor and pressure measurement data P_{X} of the water vapor, and a total system leakage X of the LSWMA, to compensate for a loss of the water vapor in the measurement data Pm of the total pressure. In this way, true pressure data Pc of the water vapor converted from the water ice in the lunar soil sample in the spectral unit is obtained.

In this embodiment, the water vapor extraction efficiency η during the vacuum heating performed by the vacuum container of the LSWMA on the lunar soil sample is measured based on a pre-experiment, and a measurement process is as follows:

A plurality of lunar soil simulants that have a same original weight m0 and different moisture contents are prepared, and the lunar soil simulants are separately transferred to the constant-volume sample receiving cup in the vacuum container of the LSWMA at a low temperature for the vacuum heating. When the vacuum heating is performed on each lunar soil simulant, the constant-volume sample receiving cup is transferred to a high-precision balance with an insulation fixture every 10 minutes, and current weight data is recorded. That is, at the first 10 minutes, the constant-volume sample receiving cup is transferred to the high-precision balance with the insulation fixture, and current weight data m1_1 is recorded; at the second 10 minutes, the constant-volume sample receiving cup is transferred to the high-precision balance with the insulation fixture, and current weight data m1_2 is recorded; at the third 10 minutes, the constant-volume sample receiving cup is transferred to the high-precision balance with the insulation fixture, and current weight data m1_3 is recorded; and at the k^{th} 10 minutes, the constant-volume sample receiving cup is transferred to the high-precision balance with the insulation fixture, and current weight data m1_k is recorded. Total vacuum heating duration t₂ of each lunar soil simulant ends only when a weight change measured by the balance is less than a minimum fluctuation of the balance, and weight data m_{end} measured by the balance when the total vacuum heating duration t₂ is reached is recorded.

In addition, in order to eliminate influence from a drift in a weight that is of the empty constant-volume sample receiving cup and recorded on the balance as heating time increases, the empty constant-volume sample receiving cup is also heated as a control group. Similarly, every 10 minutes, the empty constant-volume sample receiving cup is transferred to the balance, and weight data is recorded. That is, at the first 10 minutes, the empty constant-volume sample receiving cup is transferred to the high-precision balance with the insulation fixture, and current weight data m2_1 is recorded; at the second 10 minutes, the empty constant-volume sample receiving cup is transferred to the high-precision balance with the insulation fixture, and record current weight data m2 2 is recorded; at the third 10 minutes, the empty constant-volume sample receiving cup is transferred to the high-precision balance with the insulation fixture, and current weight data m2_3 is recorded; and at the k^{th} 10 minutes, the empty constant-volume sample receiving cup is transferred to the high-precision balance with the insulation fixture, and record current weight data m2_k is recorded.

Finally, the weight data of the constant-volume sample receiving cup is added to the weight data of each lunar soil simulant, and discrete water vapor extraction efficiency η k corresponding to the total heating duration t₂ is obtained according to a following formula: ηk=(m1_k−m2_k-m_{end})/m0, where k=1, 2, 3, . . . . Then, nonlinear fitting is performed based on the obtained discrete water vapor extraction efficiency ηk to obtain a final relationship indicating that the water vapor extraction efficiency η during the vacuum heating performed by the vacuum container on the lunar soil sample changes over time.

In this embodiment, a process for determining the relationship between the relative adsorption rate b of the pipeline for the water vapor and the pressure measurement data P_{X} of the water vapor is as follows:

A given amount of water is converted into constant-pressure water vapor of different gradients within a pressure range of 150 Pa to 1200 Pa. A microliter-scale liquid water autosampler and a high-temperature vaporizer are used jointly to inject constant-pressure water vapor of each gradient separately into the spectral unit through a pipeline, and control a temperature of a pipeline connecting the vacuum container and the spectral unit in the LSWMA to T₂. Pressure of the constant-pressure water vapor of each gradient in the spectral unit is measured by using the vacuum gauge or inverted by the spectral unit, a decrease in the pressure of the constant-pressure water vapor of each gradient in the spectral unit due to pipeline adsorption is recorded and set as an absolute adsorption amount P_{ab}. In addition, pressure when the pressure of the water vapor in the spectral unit no longer decreases after the pipeline absorption is saturated, namely equilibrium pressure P_{eq}, is recorded. The absolute adsorption amount P_{ab} is divided by the equilibrium pressure P_{eq} to obtain a relative adsorption rate b of the pipeline for the constant-pressure water vapor of each gradient, namely b=P_{ab}/P_{eq}*100. A corresponding relationship between a relative adsorption rate b obtained by fitting the pressure of the constant-pressure water vapor of each gradient and the pressure measurement data P_{X} that is of the water vapor and measured by the vacuum gauge in the spectral unit is shown in a formula (4):

$$b = 66.52 * exp ( - {P}_{X} / ( 0.566 * {T}_{2} ) ) + 8.90 \qquad ( 4 )$$

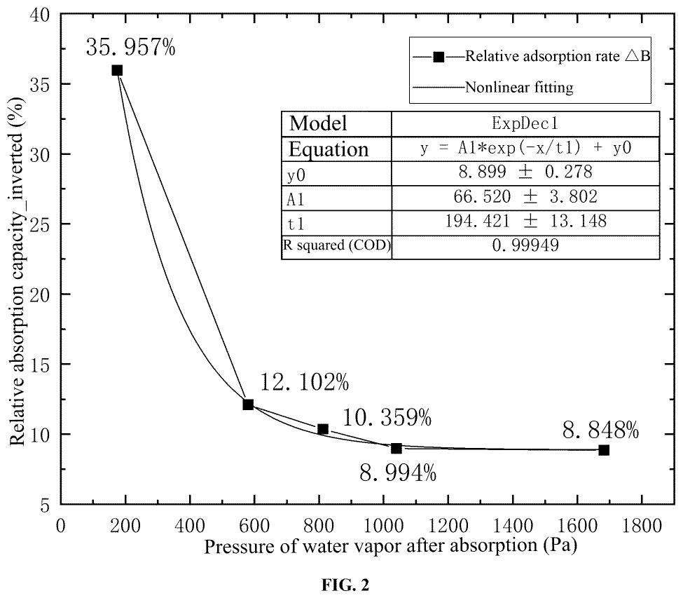

The formula (4) shows the relationship between the relative adsorption rate of the pipeline for the water vapor and the pressure measurement data of the water vapor. In this embodiment, taking T₂=35° C. as an example, a finally fitted relationship curve is shown in FIG. 2.

In this embodiment, a process for measuring the total system leakage X of the LSWMA is as follows:

Constant-pressure nitrogen is injected into a connected system constituted by the vacuum container, the pipeline, and the spectral unit in the LSWMA, a curve reflecting that pressure in the connected system decreases over time, and a static pressure rise method is used to obtain a system leakage rate Q, as shown in a formula (5):

$$Q = ( \frac{Δ P}{Δ t} ) {V}_{1} \qquad ( 5 )$$

In the formula (5), ΔP represents a measured pressure change, in units of Pa; Δt represents change time, which is change time of gas leakage in a heating and transmission process and is in units of seconds; and V₁ represents a container volume, which is a total volume of the vacuum container, the pipeline, and the spectral unit and is in units of m³.

Finally, the system leakage rate Q is multiplied by total duration t₃ of a measurement process (namely total time corresponding to the curve) to obtain the total system leakage amount X, namely X=Q×t₃.

In this embodiment, the measurement data Pm of the total pressure of the water vapor in the spectral unit is compensated and corrected according to a formula (6), and true pressure data Pc of water vapor converted from all water ice in the lunar soil in the spectral unit is obtained. The formula (6) is as follows:

$$P c = X + Pm / ( ( 1 - a ) * η * ( 1 - b ) ) \qquad ( 6 )$$

In the formula (6), X represents the total system leakage of the LSWMA; a represents the relative sublimation loss rate; η represents the water vapor extraction efficiency during the vacuum heating performed by the vacuum container on the lunar soil sample; and b represents the relative adsorption rate of the pipeline for the water vapor. During actual calculation, the relative adsorption rate b of the pipeline for the water vapor is calculated by substituting the measurement data Pm of the total pressure of the water vapor in the spectral unit into the P_{X} in the relationship between the relative adsorption rate b of the pipeline for the water vapor and the pressure measurement data of the water vapor in the formula (4).

In this embodiment, the pressure of the water vapor in the spectral unit is regarded as the true pressure data Pc of the water vapor. Then, based on a volume V₂ of the spectral unit and a measured temperature T₃ of the spectral unit, mass of the water vapor converted from the water ice in the lunar soil sample is calculated using an ideal gas law, which is mass mw of the water ice in the lunar soil sample and is in units of g. The ideal gas law is shown in a formula (7):

$$P V = n R T \qquad ( 7 )$$

In the formula (7), P represents pressure inside a container in the ideal gas law, and in this embodiment, P=Pc;
V represents a container volume in the ideal gas law, and in this embodiment, V=V₂;
T represents a temperature in the ideal gas law, and in this embodiment, T=T₃;
R represents a universal gas constant, and is 8.31447 Jmol⁻¹K⁻¹; and
n represents an amount of substance, where in this embodiment, n is molar mass of the water vapor converted from the water ice in the lunar soil sample; and assuming the molar mass of the water vapor converted from the water ice in the lunar soil sample is mₓ, and a following condition is met:

$$n = {m}_{x} = {m}_{w} / 18.015$$

18.015 represents molar mass of the water molecule, in unit of g/mol.

Therefore, the mass mw of the water ice in the lunar soil sample is shown in a formula (8):

$${m}_{w} = P c * {V}_{2} * 18.015 / ( R * {T}_{3} ) \qquad ( 8 )$$

Finally, in this embodiment, based on the mass m_{w} of the water ice in the lunar soil sample and the weight data mₛ of the lunar soil sample, mass content M of the water ice in the lunar soil is calculated, namely M=m_{w}/mₛ*100, in units of wt %.

Preferred implementations of the present disclosure are described above in detail with reference to the accompanying drawings. The embodiments described in the present disclosure are only intended to describe the preferred implementations of the present disclosure, and are not intended to limit the concept and scope of the present disclosure Various specific technical features described in the foregoing specific implementations can be combined in any suitable manner, provided that there is no contradiction. The combinations should also be regarded as the content disclosed in the present disclosure, as long as they does not violate the ideas of the present disclosure. To avoid unnecessary repetition, various possible combination modes of the present disclosure are not described separately.

The present disclosure is not limited to specific details of the above implementations. Various modifications and improvements made on the technical solutions of the present disclosure by those of ordinary skills in the art within the scope of the technical concept of the present disclosure and without departing from the design concept of the present disclosure shall fall within the claimed scope of the present disclosure. The technical content claimed by the present disclosure has been fully recorded in the claims.

What is claimed is:

1. A method for inverting a true mass content of water ice in lunar soil using a lunar soil water molecule analyzer (LSWMA), comprising: obtaining weight data of a lunar soil sample, and when a sample receiving container of the LSWMA receives the lunar soil sample, obtaining temperature data of the sample receiving container that receives only the lunar soil sample; in a process of transferring the lunar soil sample from the sample receiving container to a vacuum container by the LSWMA, obtaining a duration of the transfer process; then, performing vacuum heating on the lunar soil sample in the sample receiving container by using the vacuum container, such that the water ice in the lunar soil sample is all converted into water vapor, and when the water vapor enters a spectral unit through a pipeline, obtaining measurement data of a total pressure of the water vapor in the spectral unit;
determining a sublimation rate of the water ice in the lunar soil sample based on the temperature data of the sample receiving container, calculating a sublimation loss of the water ice in the transfer process based on the duration of the transfer process, and then estimating a relative sublimation loss rate of the water ice in the lunar soil sample in the transfer process based on the sublimation loss;
correcting the measurement data of the total pressure of the water vapor in the spectral unit based on the relative sublimation loss rate, water vapor extraction efficiency pre-measured during the vacuum heating performed by the vacuum container on the lunar soil sample, a relationship between a relative adsorption rate of the pipeline for the water vapor and pressure measurement data of the water vapor, and a total system leakage of the LSWMA, and obtaining true pressure data of the water vapor; and
calculating, based on the true pressure data of the water vapor, a mass of the water vapor converted from the water ice in the lunar soil sample to obtain a mass of the water ice in the lunar soil sample, and calculating a mass content of the water ice in the lunar soil sample based on the mass of the water ice and the weight data of the lunar soil sample.

2. The method for inverting the true mass content of the water ice in lunar soil using the LSWMA according to claim 1, wherein based on the temperature data of the sample receiving container, the sublimation rate S of the water ice is calculated using a calculation formula for a sublimation rate of pure water ice, as shown in a formula (1):
$$S = {e}_{s a t , i} ( T ) \times \sqrt{\frac{{M}_{w}}{2 π R T}} \times exp ( \frac{2 {M}_{w} {σ}_{i}}{{ρ}_{i} R T} ) \qquad ( 1 )$$
wherein in the formula (1), e_{sat,i}(T) represents saturated vapor pressure of water, and is obtained by calculating a temperature of the lunar soil sample based on the temperature data of the sample receiving container and then substituting the temperature of the lunar soil sample into an empirical formula shown in a formula (2):
$${e}_{s a t , i} = exp ( 9.550426 - \frac{5 723.265}{T} + 3.53068 ln T - 0.00728332 T ) ; \qquad ( 2 )$$
M_{W} represents molar mass of a water molecule, and is 18.015×10⁻³ kg·mol⁻¹;
R represents a universal gas constant, and is 8.31447 Jmol⁻¹K⁻¹;
$$exp ( \frac{2 {M}_{w} {σ}_{i}}{{ρ}_{i} R T} )$$
represents a correction term for saturated vapor pressure generated by the water ice due to surface tension under a true condition, wherein σᵢ represents surface tension on a pure ice/vapor interface, with a value of 0.109 Jm⁻²; and ρᵢ represents a density of the water ice at a sublimation temperature, and a calculation formula for the ρᵢ is shown in a formula (3):
$${ρ}_{i} = 916.7 - 0.1 \; 75 ( T - 273.15 ) - 5.0 \times 1 {0}^{- 4} ( T - 273.15 ) ; \qquad ( 3 )$$
in the above formulas (1), (2), and (3), T is taken as the temperature of the lunar soil sample and calculated based on the temperature data of the sample receiving container;
then, a theoretical sublimation loss of the water ice in the transfer process is calculated according to a following formula: A=S×t₁, wherein t₁ represents the duration of the transfer process;
next, the theoretical sublimation loss is corrected based on a correction coefficient k, wherein the correction coefficient k is related to a particle size and a pore of the lunar soil sample and obtained from a pre-experiment, and an actual sublimation loss is obtained according to a following formula: A₁=A×k; and
finally, based on the actual sublimation loss A₁ and an original water amount m1 corresponding to the correction coefficient k during the pre-experiment, the relative sublimation loss rate is calculated according to a following formula:
$$a = \frac{A 1}{m 1} .$$

3. The method for inverting the true mass content of the water ice in lunar soil using the LSWMA according to claim 2, wherein a process for determining the correction coefficient k based on an experiment is as follows: during the experiment, taking a plurality of lunar soil simulants with different porosity and particle sizes and a same water content of mass m1, placing the plurality of lunar soil simulants in the sample receiving container, and obtaining sublimation losses of each of the plurality of lunar soil simulants within a plurality of different temperature ranges in the sample receiving container; and obtaining sublimation losses of pure water ice with mass of m1 within a plurality of corresponding temperature ranges in the sample receiving container, and determining the correction coefficient k based on the sublimation losses of the plurality of lunar soil simulants and the sublimation losses of the pure water ice.

4. The method for inverting the true mass content of the water ice in lunar soil using the LSWMA according to claim 1, wherein when the water vapor extraction efficiency during the vacuum heating performed by the vacuum container on the lunar soil sample in the sample receiving sample is measured, a plurality of lunar soil simulants that have a same original weight but different moisture contents are taken and separately placed in a sample receiving container of the vacuum container for heating, weight data of each of the plurality of lunar soil simulants during the heating is recorded, and then the weight data of each of the plurality of lunar soil simulants is nonlinearly fitted to obtain the water vapor extraction efficiency during the vacuum heating performed by the vacuum container on the lunar soil sample.

5. The method for inverting the true mass content of the water ice in lunar soil using the LSWMA according to claim 4, wherein when the water vapor extraction efficiency is measured, weight data of the sample receiving container in empty state during the heating is also recorded, and after a weight change of the sample receiving container in empty state due to the heating is deducted, nonlinear fitting is performed to obtain the water vapor extraction efficiency during the vacuum heating performed by the vacuum container on the lunar soil sample.

6. The method for inverting the true mass content of the water ice in lunar soil using the LSWMA according to claim 1, wherein when the relationship between the relative adsorption rate of the pipeline for the water vapor and the pressure measurement data of the water vapor is determined, a given amount of water is taken and converted into constant-pressure water vapor of a plurality of gradients, and constant-pressure water vapor of each of the plurality of gradients is separately injected into the spectral unit through the pipeline; a pressure change of the constant-pressure water vapor of each of the plurality of gradients in the spectral unit due to pipeline adsorption, as well as pressure that is of the water vapor in the spectral unit and no longer decreases after the pipeline adsorption is saturated, namely an equilibrium pressure, are recorded, and a relative adsorption rate of the constant-pressure water vapor of each of the plurality of gradients is obtained based on the pressure change and the equilibrium pressure; and then, based on fitted pressure of the constant-pressure water vapor of the plurality of gradients, a corresponding relationship between the relative adsorption rate and pressure data that is of the water vapor and measured by a vacuum gauge for the spectral unit is obtained, that is, the relationship between the relative adsorption rate of the pipeline for the water vapor and the pressure measurement data of the water vapor is obtained.

7. The method for inverting the true mass content of the water ice in lunar soil using the LSWMA according to claim 1, wherein when the total system leakage of the LSWMA is measured, a constant-pressure gas is injected into a connected system constituted by the vacuum container, the pipeline and the spectral unit in the LSWMA, a curve reflecting that pressure in the spectral unit changes over time is recorded, then a system leakage rate is obtained by using a static pressure rise method based on the curve, and finally, the total system leakage is calculated based on the system leakage rate and total time corresponding to the curve.

8. The method for inverting the true mass content of the water ice in lunar soil using the LSWMA according to claim 1, wherein the measurement data of the total pressure of the water vapor in the spectral unit is corrected according to a following formula (6):
$$P c = X + Pm / ( ( 1 - a ) * η * ( 1 - b ) ) \qquad ( 6 )$$
wherein in the formula (6), Pc represents the true pressure data of the water vapor; Pm represents the measurement data of the total pressure of the water vapor in the spectral unit; X represents the total system leakage of the LSWMA; a represents the relative sublimation loss rate; η represents the water vapor extraction efficiency during the vacuum heating performed by the vacuum container on the lunar soil sample; b represents the relative adsorption rate of the pipeline for the water vapor, wherein the relative adsorption rate b of the pipeline for the water vapor is calculated by substituting the measurement data of the total pressure of the water vapor in the spectral unit into the relationship between the relative adsorption rate of the pipeline for the water vapor and the pressure measurement data of the water vapor.

9. The method for inverting the true mass content of the water ice in lunar soil using the LSWMA according to claim 1, wherein based on the true pressure data of the water vapor, a volume of the spectral unit and a measured temperature of the spectral unit, the mass of the water vapor converted from the water ice in the lunar soil sample is calculated using an ideal gas law.
